(19) United States
(12) Patent Application Publication
(10) Pub. No.: US 2026/0223944 A1
(43) Pub. Date: Aug. 6, 2026

(54) MULTI-LEVEL AUTHORIZATION FOR AN AEROSOL-GENERATING DEVICE

(71) Applicant: Philip Morris Products S.A., Neuchatel (CH)

(72) Inventor: Sander BROUWER, Lausanne (CH)

(73) Assignee: Philip Morris Products S.A., Neuchatel (CH)

(21) Appl. No.: 19/122,101

(22) PCT Filed: Oct. 25, 2023
(86) PCT No.: PCT/EP2023/079814

§ 371 Date: Apr. 17, 2025

(30) Foreign Application Priority Data
Oct. 25, 2022 (EP) 22203485

(51) Int. Cl. A24F 40/49; A24F 40/51; A24F 40/53; G06F 21/32; G06V 10/82; G06V 40/16

(52) U.S. Cl. CPC A24F 40/49; A24F 40/51; A24F 40/53; G06F 21/32; G06V 10/82; G06V 40/172; G06V 40/174

(57) ABSTRACT

An aerosol-generating system is provided, including: an aerosol-generating device configured to generate an aerosol from an aerosol-forming substrate; an image acquisition sensor configured to capture at least one image of a user of the aerosol-generating device; an acoustic sensor configured to detect a sound generated based on a respiration of the user; and at least one processor configured to control, based on the at least one image of the user and the detected sound, at least one of: an access of the user to the aerosol-generating device, an amount of a substance generated from the aerosol-forming substrate, and an amount aerosol generated from the aerosol-forming substrate.

The present disclosure relates to an aerosol-generating system. In particular, the disclosure relates to a system for controlling an aerosol-generating device.

Aerosol-generating devices may comprise an electrically operated heat source that is configured to heat an aerosol-forming substrate to produce an aerosol, such as a nicotine-containing aerosol. However, such electronic devices should not be accessed by unauthorized users. Accordingly, there is a need for a system that would enable only authorized users to access the electronic device.

Usage of an aerosol-generating device may have an effect on a user under certain biological conditions of the user. For example, it may be preferable to limit usage of an aerosol-generating device for a user having a lung-related disease. Accordingly, there is a need for a system that limits the usage of the electronic device based on a biological condition of the user.

An aerosol-generating device may be used by a plurality of users. However, different users may require or prefer a different quantity or composition of aerosol formed from an aerosol-forming substrate. Some users compared to other users may require or prefer a different flavor content or a different medicament content compared to another content of a substance. For example, some of the plurality of users may require or prefer to consume an increased content of a first substance compared to a content of a second substance of the aerosol-forming substrate disposed in the electronic device. Accordingly, there is a need for a system that selectively controls a dosing of at least one substance or the aerosol generated from the aerosol-forming substrate.

According to an aspect of the present invention, there is provided an aerosol-generating system, comprising an aerosol-generating device configured to generate an aerosol from an aerosol-forming substrate, an image acquisition sensor configured to capture at least one image of a user of the aerosol-generating device, an acoustic sensor configured to detect a sound generated based on a respiration of the user, and at least one processor, configured to control, based on at least one of the at least one image of the user and the detected sound, at least one of (i) an access of the user to the aerosol-generating device, (ii) an amount of a substance generated from the aerosol-forming substrate, and (iii) an amount of aerosol generated from the aerosol-forming substrate. The aerosol-forming substrate may be disposed in the aerosol-generating device and/or engaged with the aerosol-generating device.

The amount of the substance or aerosol generated may be controlled by, for example, controlling a temperature profile of a heater of the aerosol-generating device. Controlling a temperature profile of the heater may comprise one or both of controlling a temperature of the heater over time, and controlling a power supplied to the heater over time. The amount of the substance of the aerosol may be controlled by, for example, selecting one of a plurality of predetermined heater temperature profiles. Thus, one of a plurality of predetermined heater temperature profiles may be selected based on at least one of the at least one image of the user and the detected sound.

By providing an aerosol-generating system comprising an image acquisition sensor and an acoustic sensor, an improved aerosol-generating system is provided for controlling an aerosol-generating device in a safe and secure manner. Specifically, an aerosol-generating system comprising an image acquisition sensor and an acoustic sensor allows the restriction of access to the aerosol-generating device to only authorized users and provides for multiple levels of authorization. Additionally, or alternatively, the aerosol-generating system provides for customized control of the aerosol-generating device based on data from the image acquisition sensor and the acoustic sensor, where the acoustic sensor can be used to determine a biological condition of the user. This allows modifying, for example limiting, usage of the aerosol-generating device based on the biological condition of the user. For example, the access of the user to the aerosol-generating device may be limited to a predetermined number of puffs (e.g. within a predetermined time period), or a predetermined usage duration.

Furthermore, the aerosol-generating system may allow selective control of a dosing of at least one substance content, such as a flavor content or medicament content of an aerosol-forming substrate, or selective control of a dosing duration of the aerosol-forming substrate, which may be based on a user profile of an authorized user.

The at least one processor may be configured to determine, based on the detected sound, a biological status, such as a lung-related condition or a level of bronchitis, of the user. Additionally or alternatively, the at least one processor may be configured to determine, based on the at least one image of the user, at least one of an identity, a physical state and an emotional state of the user. This allows the control of the aerosol-generating device based on the biological status and the at least one of the identity, the physical state and the emotional state of the user. To further increase security, the at least one processor may be configured to verify the identity of the user based on the biological status of the user.

According to another aspect of the present invention, there is provided a method comprising capturing, by an image acquisition sensor, at least one image of a user; detecting, by an acoustic sensor, a sound generated based on a respiration of the user; and controlling, based on at least one of the at least one image of the user and the detected sound, at least one of an access of the user to an aerosol-generating device, an amount of a substance generated from an aerosol-forming substrate, and amount of aerosol generated from an aerosol-forming substrate.

By controlling an access of the user to the aerosol-generating device based on the at least one image of the user and the detected sound, an improved aerosol-generating system is provided that provides for two-level authentication via the image acquisition sensor and the acoustic sensor.

As used herein, the term “aerosol-generating device” refers to a device that interacts with an aerosol-forming substrate to generate an aerosol. An aerosol-generating device may interact with one or both of an aerosol-generating article comprising an aerosol-forming substrate, and a cartridge comprising an aerosol-forming substrate. In some examples, the aerosol-generating device may heat the aerosol-forming substrate to facilitate release of volatile compounds from the substrate. An electrically operated aerosol-generating device may comprise an atomizer, such as an electric heater, to heat the aerosol-forming substrate to form an aerosol.

As used herein, the term “access to the aerosol-generating device” may refer to access to operation of the aerosol-generating device to generate an aerosol. Thus, preventing access to the aerosol-generating device may refer to preventing operation of the aerosol-generating device to generate an aerosol.

As used herein, the term “aerosol-forming substrate disposed in and/or engaged with the aerosol-generating device” refers to the combination of an aerosol-generating device with an aerosol-forming substrate. When the aerosol-forming substrate forms part of an aerosol-generating article, the aerosol-forming substrate disposed in the aerosol-generating device refers to the combination of the aerosol-generating device with the aerosol-generating article. The aerosol-forming substrate and the aerosol-generating device may cooperate to generate an aerosol.

As used herein, the term “aerosol-forming substrate” refers to a substrate capable of releasing volatile compounds that can form an aerosol. The volatile compounds may be released by heating the aerosol-forming substrate. As an alternative to heating, in some cases, volatile compounds may be released by a chemical reaction or by a mechanical stimulus, such as ultrasound. The aerosol-forming substrate may be solid or may comprise both solid and liquid components. An aerosol-forming substrate may be part of an aerosol-generating article.

As used herein, the term “aerosol-generating article” refers to an article comprising an aerosol-forming substrate that is capable of releasing volatile compounds that can form an aerosol. The aerosol may comprise nicotine. An aerosol-generating article may be disposable. An aerosol-generating article comprising an aerosol-forming substrate comprising tobacco may be referred to herein as a tobacco stick.

An aerosol-forming substrate may comprise nicotine. An aerosol-forming substrate may comprise tobacco, for example a tobacco-containing material containing volatile tobacco flavor compounds, which are released from the aerosol-forming substrate upon heating. In preferred embodiments an aerosol-forming substrate may comprise homogenized tobacco material, for example cast leaf tobacco. The aerosol-forming substrate may comprise both solid and liquid components. The aerosol-forming substrate may comprise a tobacco-containing material containing volatile tobacco flavor compounds, which are released from the substrate upon heating. The aerosol-forming substrate may comprise a non-tobacco material. The aerosol-forming substrate may further comprise an aerosol former. Examples of suitable aerosol formers are glycerin and propylene glycol.

The invention is defined in the claims. However, a non-exhaustive list of non-limiting examples is provided below. Any one or more of the features of these examples may be combined with any one or more features of another example, embodiment, or aspect described herein.

Example Ex1: An aerosol-generating system, comprising an aerosol-generating device configured to generate an aerosol from an aerosol-forming substrate, an image acquisition sensor configured to capture at least one image of a user of the aerosol-generating device, an acoustic sensor configured to detect a sound generated on the basis of the respiration of the user, and at least one processor, configured to control, based on at least one of the at least one image of the user and the detected sound, at least one of an access of the user to the aerosol-generating device, an amount of a substance generated from the aerosol-forming substrate, and an amount of aerosol generated from the aerosol-forming substrate.

Example Ex2: The aerosol-generating system according to example Ex1, wherein the at least one processor is configured to determine, based on the detected sound, a biological status of the user.

Example Ex3: The aerosol-generating system according to example Ex2, wherein the biological status comprises at least one of a lung-related condition and a level of bronchitis of the user.

Example Ex4: The aerosol-generating system according to one of examples Ex2 and Ex3, wherein the at least one processor is configured to determine, based on the at least one image of the user, at least one of an identity, a physical state and an emotional state of the user.

Example Ex5: The aerosol-generating system according to example Ex4, wherein the at least one of the access of the user to the aerosol-generating device, the amount of the substance generated from the aerosol-forming substrate, and an amount of aerosol generated from the aerosol-forming substrate is controlled based on the biological status and the at least one of the identity, the physical state and the emotional state of the user.

Example Ex6: The aerosol-generating system according to one of examples Ex4 and Ex5, wherein the at least one processor is configured to determine a user profile based on at least one of the biological status and the at least one of the identity, the physical state and the emotional state of the user, and wherein the amount of the substance and/or aerosol generated from the aerosol-forming substrate is controlled in accordance with the user profile.

Example Ex7: The aerosol-generating system according to one of examples Ex4 to Ex6, wherein the at least one processor is configured to verify the identity of the user based on the biological status of the user.

Example Ex8: The aerosol-generating system according to one of examples Ex4 to Ex7, wherein at least one of the biological status and the at least one of an identity, a physical state and an emotional state of the user is determined by using a neural network.

Example Ex9: The aerosol-generating system according to one of examples Ex1 to Ex8, wherein the at least one processor is configured to control the acoustic sensor to detect the sound based on an analysis of the at least one image of the user, wherein the analysis of the at least one image of the user comprises a determination whether the user is authorized.

Example Ex10: The aerosol-generating system according to one of examples Ex1 to Ex9, wherein the at least one processor is configured to control the access of the user to the aerosol-generating device by limiting usage of the aerosol-generating device to a predetermined number of puffs, such as a predetermined number of puffs in a predetermined time period, or a predetermined usage duration.

Example Ex11: The aerosol-generating system according to one of examples Ex1 to Ex10, wherein the controlling the amount of a substance generated from the aerosol-forming substrate comprises increasing the amount of one substance content of the aerosol-forming substrate with respect to another substance content of the aerosol-generating substrate.

Example Ex12: The aerosol-generating system according to one of examples Ex1 to Ex11, wherein the aerosol-generating system comprises at least one of a temperature sensor configured to measure a temperature of the user, for example a temperature of skin in contact with a portion of the aerosol-generating system (such as the aerosol-generating device), and an inertial motion unit, IMU, sensor, wherein data of the IMU sensor is used to determine a usage behavior of the user, and wherein the at least one of the access of the user to the aerosol-generating device, and the amount of the substance/aerosol generated from the aerosol-forming substrate is controlled based on the at least one of the at least one image of the user and the detected sound, and at least one of the usage behavior of the user and the temperature.

Example Ex13: The aerosol-generating system according to one of examples Ex1 to Ex12, wherein the at least one processor is configured to determine whether the user is authorized to use the aerosol-generating device based on the at least one image of the user, and wherein the at least one processor is configured to, when the user is unauthorized, at least one of lock the aerosol-generating device to prevent usage of the aerosol-generating device from the unauthorized user, and provide an alert to an authorized user of the aerosol-generating device, for example by providing the alert to a linked mobile phone, computer or other device of the authorized user.

Example Ex14: The aerosol-generating system according to example Ex13, wherein the alert comprises information associated with the unauthorized user attempt on the aerosol-generating device.

Example Ex15: The aerosol-generating system according to example Ex13 or Ex14, wherein the alert comprises at least one of: an audible alert, a visual alert, an audio-visual alert, or a vibratory alert.

Example Ex16: The aerosol-generating system according to one of examples Ex1 to Ex15, wherein the at least one processor is configured to control the image acquisition sensor to capture the at least one image of the user based on an analysis of the sound.

Example Ex17: The aerosol-generating system according to example Ex16, wherein the analysis of the sound comprises a determination whether the user is in a physical condition to use the aerosol-generating device.

Example Ex18: The aerosol-generating system according to example Ex4, or any preceding examples when dependent on example Ex4, wherein the at least one of an identity, a physical state and an emotional state of the user is determined by determining a plurality of different features of a face of the user.

Example Ex19: The aerosol-generating system according to one of examples Ex1 to Ex18, wherein the substance comprises at least one of a flavor content, a nicotine content and a medicament content.

Example Ex20: The aerosol-generating system according to example Ex4, or any preceding examples when dependent on example Ex4, wherein the emotional state of the user comprises at least one of a happy emotion, a sad emotion, an angry emotion, a calm emotion, a fearful emotion, a neutral emotion, an excited emotion, a confused emotion, a stressed emotion, a disgusted emotion, a surprised emotion, an excitement emotion, and a scared emotion.

Example Ex21: The aerosol-generating system according to example Ex4, or any preceding examples when dependent on example Ex4, wherein the physical state of the user comprises at least one of an age, a sex, a gesture relating to an authorization of the user, a tired state, a drunken state and a stressed state.

Example Ex22: The aerosol-generating system according to one of examples Ex2 and Ex3, or any preceding examples when dependent on example Ex2 or Ex3,wherein the biological status of the user is determined based on at least one voice parameter obtained from the sound generated on the basis of the respiration of the user, wherein the at least one voice parameter comprises at least one of a loudness parameter, an intonation parameter, an intensity of overtones, a voice modulation parameter, a pitch parameter, a tone parameter, a rate-of-speech parameter, a voice quality parameter, a phonetic parameter, a pronunciation parameter, a prosody parameter, a timbre parameter, and one or more psychoacoustic parameters.

Example Ex23: The aerosol-generating system according to one of examples Ex1 to Ex22, wherein the image acquisition sensor is comprised in the aerosol-generating device.

Example Ex24: The aerosol-generating system according to one of examples Ex1 to Ex22, wherein the aerosol-generating system comprises an image acquisition device, such as a mobile phone or computer, comprising the image acquisition sensor, wherein the image acquisition device is remote from the aerosol-generating device, and wherein the image acquisition device and the aerosol-generating device are connected to a wireless network.

Example Ex25: The aerosol-generating system according to one of examples Ex1 to Ex24, wherein the image acquisition sensor is configured to capture depth information of the user from at least one viewpoint.

Example Ex26: The aerosol-generating system according to one of examples Ex1 to Ex25, wherein the image acquisition sensor comprises one of a Light Detection and Ranging (LIDAR) sensor, a wide-angle camera, an action camera, a closed-circuit television (CCTV) camera, a camcorder, a digital camera, camera phones, a time-of-flight (ToF) camera, and a night-vision camera.

Example Ex27: The aerosol-generating system according to one of examples Ex1 to Ex26, wherein the acoustic sensor comprises one of a recorder, an electric microphone, a dynamic microphone, a carbon microphone, a piezoelectric microphone, a fiber microphone, and a micro-electro-mechanical-systems, MEMS, microphone.

Example Ex28: The aerosol-generating system according to one of examples Ex1 to Ex27, wherein the at least one processor is configured to control the image acquisition sensor to capture the at least one image of the user.

Example Ex29: The aerosol-generating system according to one of examples Ex1 to Ex28, wherein the at least one processor is configured to control the acoustic sensor to detect the sound.

Example Ex30: The aerosol-generating system according to example Ex4, or any preceding examples when dependent on example Ex4, wherein the aerosol-generating system is configured to compare information associated with the at least one image of the user with information associated with one or more pre-stored images to determine different facial features associated with the user, and wherein the determined different facial features are used to determine the at least one of an identity, a physical state and an emotional state of the user.

Example Ex31: The aerosol-generating system according to example Ex30, wherein the information associated with the one or more pre-stored images is integrally stored in the aerosol-generating device or remotely retrieved from a server.

Example Ex32: The aerosol-generating system according to one of examples Ex2 and Ex3, or any preceding examples when dependent on example Ex2 or Ex3, wherein the aerosol-generating system is configured to compare information associated with the biological status with information associated with a pre-stored biological status, to determine a level of a lung-related condition of the user, and wherein the determined the level of the lung-related condition is used to control the at least one of an access of the user to the aerosol-generating device, and an amount of a substance and/or an aerosol generated from the aerosol-forming substrate.

Example Ex33: The aerosol-generating system according to example Ex32, wherein the information associated with the pre-stored biological status is integrally stored in the aerosol-generating device or remoted retrieved from a server.

Example Ex34: The aerosol-generating system according to example Ex12, or any preceding examples when dependent on example Ex12, wherein the usage behavior comprises a pattern of usage of the aerosol-generating device.

Example Ex35: The aerosol-generating system according to example Ex12, wherein the IMU sensor comprises an accelerometer and a gyroscope, which is configured to measure an angular rate, a force and positional orientations of the aerosol-generating device during usage of the aerosol-generating device.

Example Ex36: The aerosol-generating system according to example Ex12, or any preceding examples when dependent on example Ex12, wherein the at least one of the access of the user to the aerosol-generating device, and the amount of the substance and/or aerosol content of the aerosol-forming substrate is controlled based on the usage behavior and the at least one of the at least one image of the user and the detected sound.

Example Ex37: An aerosol-generating device of an aerosol-generating system according to any of examples Ex1 to Ex36, wherein the device comprises any one, two or all of the image acquisition sensor, the acoustic sensor, and the at least one processor.

Example Ex38: The aerosol-generating system according to any of examples Ex1 to Ex37, further comprising the aerosol-forming substrate.

Example Ex39: The aerosol-generating system according to any of examples Ex1 to Ex38, wherein the aerosol-forming substrate may be disposed in the aerosol-generating device and/or engaged with the aerosol-generating device.

Example Ex40: A method comprising capturing, by an image acquisition sensor, at least one image of a user of an aerosol-generating device; detecting, by an acoustic sensor, a sound generated based on a respiration of the user; and controlling, based on at least one of the at least one image of the user and the detected sound, at least one of an access of the user to the aerosol-generating device, an amount of a substance content of an aerosol-forming substrate, and an amount of aerosol generated from an aerosol-forming substrate.

It will be appreciated that any of the above examples Ex2-38 regarding the system can formulated as a method according to example Ex39.

Examples will now be further described with reference to the figures in which:

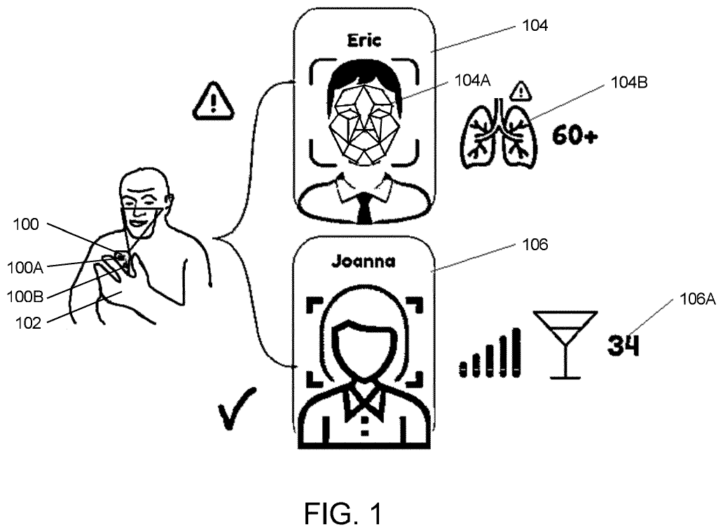

FIG. 1 shows a schematic illustration of an aerosol-generating system; and

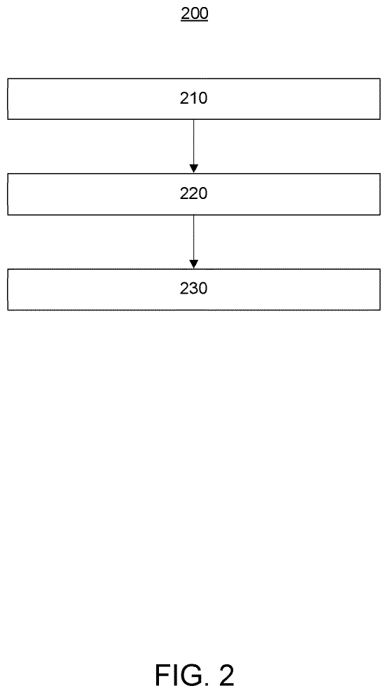

FIG. 2 is a flow diagram showing a method of controlling an aerosol-generating device.

FIG. 1 illustrates an aerosol-generating system. The aerosol-generating system comprises an aerosol-generating device 100 configured to generate an aerosol from an aerosol-forming substrate engaged with the aerosol-generating device 100. The aerosol-generating system comprises an image acquisition sensor 100A configured to capture at least one image of a user 102. The aerosol-generating system further comprises an acoustical sensor 100B configured to detect a sound generated based on a respiration of the user 102.

At least one of the image acquisition sensor 100A and the acoustical sensor 100B may be comprised in the aerosol-generating device 100. Alternatively, at least one of the image acquisition sensor 100A and the acoustical sensor 100B may be remote from the aerosol-generating device 100.

The image acquisition sensor 100A may be part of an image acquisition device. The image acquisition sensor 100A, the acoustical sensor 100B and the aerosol-generating device 100 may all be in connected via a network. The network may be a wireless network.

The aerosol-generating device 100 may be a heat-not-burn (HNB) device. The aerosol-generating system may be used to detect a user profile associated with a user 102. Based on the detected user profile, the aerosol-generating system may be configured to control at least one of an activation of the aerosol-generating device, and a dosing of one or more substances (such as, a nicotine content, a flavor content or even other medicament content) for the user.

The image acquisition sensor 100A may be configured to capture an image or a plurality of images of the user 102 using the aerosol-generating device 100. The image acquisition sensor 100A may be configured to capture depth information or a plurality of depth values of the user from a single viewpoint or a plurality of viewpoints. Examples of the image acquisition sensor 100A may include a LIDAR (Light Detection and Ranging) sensor, a wide-angle camera, an action camera, a closed-circuit television (CCTV) camera, a camcorder, a digital camera, camera phones, a time-of-flight (ToF) camera, a night-vision camera, an image sensor, and/or other image capturing devices.

The one or more captured images may indicate physical information associated with the user 102 recognized in at least one frame of the one or more captured images. The physical information may indicate at least one of an age of the user, motion information of the user, or a gesture associated with the user. Such physical information about the user may be calculated based on different features, such as a size, proportions, distances and geometry, related to the face of the user 102, where such features may be estimated from the captured images of the user.

The one or more captured images may also be utilized to determine a facial state or expression of the user 102. The facial state or expression may indicate one or more motions or positions of muscles of a face of the user 102, where the facial expressions may manifest an emotion. The muscles of the face may move the skin of the user, which may create facial lines/folds, or may cause the movement of facial features, such as mouth, head, nose, eye, and eyebrows of the user. The aerosol-generating system may be configured to determine the state of the user based on the determined facial state or expression of the user 102. The state of the user 102 may be tired, drunk, stressed, etc. The aerosol-generating system may be further configured to determine the emotional state of the user 102 based on the determined facial state or expression of the user 102. The emotional state of the user 102 may be a happy emotion, a sad emotion, an angry emotion, a calm emotion, a fear emotion, a neutral emotion, an excited emotion, a confused emotion, a stressed emotion, a disgusted emotion, a surprised emotion, an excitement emotion, or a scared emotion.

In an example, the aerosol-generating device may include an inbuilt neural-network-based processor that is configured to communicate with the image acquisition sensor 100A for basic interactions associated with the image acquisition sensor 100A. The neural network may include electronic data, such as, for example, a software program, code of the software program, libraries, applications, scripts, or other logic or instructions for execution by a processing device, such as the aerosol-generating system or the aerosol-generating device 100. The neural network may include code and routines configured to enable a computing device, such as the aerosol-generating device 100 to perform one or more operations for classification of one or more inputs. Further, the neural network may also be implemented using hardware including a processor, a microprocessor (e.g., to perform or control performance of one or more operations), a field-programmable gate array (FPGA), or an application-specific integrated circuit (ASIC). Alternatively, in some aspects, the neural network may be implemented using a combination of hardware and software. In another example, the aerosol-generating device 100 is configured to interact with a smartphone for advanced interactions associated with the image acquisition sensor 100A. For example, the image acquisition sensor 100A may be comprised in a smartphone or other handheld computing device and the smartphone may be configured to process the images obtained from the image acquisition sensor 100A. The smartphone may be configured to determine, based on at least one image of the user 102, at least one of an identity, a physical state and an emotional state of the user 102. The at least one of an identity, a physical state and an emotional state of the user 102 may be transmitted to the aerosol-generating device 100 for further processing. Alternatively, the smartphone may be configured to receive further information, such as a biological status, for further processing. Results of such further processing may be transmitted to the aerosol-generating device 100 for controlling the aerosol-generating device 100. In one aspect, the smartphone obtains the at least one image of the user 102 and transmits the at least one image of the user 102 to the aerosol-generating device 100 for further processing.

The bio-acoustic sensor 100B may be configured to capture an audio signal from the lungs of the user 102. The bio-acoustic sensor 100B may further be configured to convert the captured audio signal into an electrical signal to determine the biological state of the user. For example, the biological state of the user may correspond to a level of bronchitis or other lung or breathing related disease of the user 102. The captured audio signal may include a plurality of voice parameters, such as a loudness parameter, an intonation parameter, an intensity of overtones, a voice modulation parameter, a pitch parameter, a tone parameter, a rate-of-speech parameter, a voice quality parameter, a phonetic parameter, a pronunciation parameter, a prosody parameter, a timbre parameter, and one or more psychoacoustic parameters, which may be converted into the electrical signal to determine the biological state (for example, the level of bronchitis) of the user. Examples of the bio-acoustic sensor 100B may include a recorder, an electric microphone, a dynamic microphone, a carbon microphone, a piezoelectric microphone, a fiber microphone, a (micro-electro-mechanical-systems) MEMS microphone, or other microphones known in the art.

In operation, the aerosol-generating device 100 may control the image acquisition sensor 100A to capture the plurality of images of the user 102. The aerosol-generating device 100 may compare information associated with the one or more captured images with information associated with pre-stored images to determine different facial features associated with the user 102. The information associated with the pre-stored images may be integrally stored in the aerosol-generating device 100 or remotely retrieved from a server (for example, a cloud server) or a smartphone, via a suitable communication network (for example, via a Wireless Fidelity (Wi-Fi) network). Based on the determined facial features, the aerosol-generating device 100 may be configured to estimate a physical parameter associated with the user. For example, the physical parameter may include the age or the emotional state of the user. The aerosol-generating device 100 may further control the bio-acoustic sensor 100B to detect acoustic behavior (such as a sound that may be generated based on respiration of the user) of the lungs of the user. Based on the detected acoustic behavior of the lungs, the aerosol-generating device 100 may estimate a biological state (for example, a level of bronchitis or a level of lung-related disease) of the user 102. The aerosol-generating device 100 may compare information associated with the estimated level of a biological state with information associated with pre-stored levels of biological states to determine a biological level (such as a level of lung condition) of the user 102. The information associated with the pre-stored levels of biological states may be integrally stored in the aerosol-generating device 100 or retrieved from a remote server, via a suitable communication network. Based on the estimated physical parameters, such as an age, a stress level, a level of tiredness, or an emotional state, and the biological state, such as a level of lung-related disease, of the user, the aerosol-generating device 100 may be configured to control at least one of an activation of the aerosol-generating device or a dosing of one or more substances (such as a nicotine content, a flavor content or even other medicament content) for the user.

For example, the image acquisition sensor 100A may capture a plurality of images of a first user 104. The aerosol-generating system may compare the captured plurality of images with the pre-stored images to determine different features 104A associated with the first user 104. The different features may correspond to at least one of an age, a sex, an identity, or a gesture that relates to an authorization of the first user 104. Based on the determined features, if the first user 104 (such as a person below an age threshold) is not an authorized user, the aerosol-generating device may be locked and/or turned off to prevent usage from the unauthorized user. The aerosol-generating system may also generate an alert to an authorized user. For example, the alert may be provided to an authorized user or a device associated with the authorized user, such as a parent's mobile phone or an owner's mobile phone. The alert may comprise information about the unauthorized usage of the aerosol-generating device by the unauthorized user. The alert may include information associated with an unauthorized user attempt on the aerosol-generating device. The alert may include at least one of an audible alert, a visual alert, an audio-visual alert, or a vibratory alert. In another example, based on the determined features, if the captured images indicate that the authorized user is tired, drunk, stressed, or emotionally unstable, such that the user is in a sad, crying, or scared condition, the aerosol-generating device may automatically turn off the aerosol-generating device 100 based on the condition of the user.

If a second user is an authorized user, such as a person above an age threshold owner of the device 100, the aerosol-generating device 100 may control the bio-acoustic sensor 100B to detect an acoustic behavior, such as a sound that may be generated based on respiration of the second user's lungs. Based on the detected acoustic behavior of the lungs, the aerosol-generating device may estimate the biological state. For example, the aerosol-generating device may estimate the level of bronchitis of the second user. The estimated level of biological state may be compared with the pre-stored levels of biological states, to determine the biological level of the second user. In an example, if the second user does not meet a required biological level, for example, if the lung condition of the user is unstable, the aerosol-generating device may lock and/or turn off to prevent usage from the second user, who may then be regarded as an unauthorized user. The aerosol-generating device may also control at least one of a consumption amount of the aerosol-generating device, or a consumption duration of the aerosol-generating device. In another example, if the second user meets the required biological level, for example, if the lung condition of the user is stable, the aerosol-generating device may unlock to allow usage from the authorized user.

This sequence of the operations of the image acquisition sensor and the bio-acoustic sensor is merely provided as an example. The sequence of operations may be modified, reversed, or removed to achieve the same objective. For example, the bio-acoustic sensor may detect the acoustic behavior of the user prior to the image acquisition sensor, or the bio-acoustic sensor may detect the acoustic behavior of the user concurrently with the image detection of the user from the image acquisition sensor.

In an example, the image acquisition sensor 100A may capture one or more images of a third user 106. The aerosol-generating device may compare the one or more captured images with the pre-stored images to determine different features associated with the third user. The different features may correspond to at least one of an age, a sex, an identity or a gesture that relates to an authorization of the third user. Based on the determined features, if the third user is an authorized user, the aerosol-generating device may unlock and/or turn on to grant usage access of the aerosol-generating device to the authorized user (i.e., the third user, such as a recognized owner above an age threshold of the aerosol-generating device).

The aerosol-generating system controls the bio-acoustic sensor to detect an acoustic behavior, such as an indication corresponding to a sound that may be generated on the basis of the respiration of the third user, or the condition of the lungs of the third user. Based on the detected acoustic behavior of the lungs, the aerosol-generating device may estimate the biological state (for example, the level of bronchitis) of the third user. The estimated level of biological state is compared with the pre-stored biological state, to determine the biological level of the third user. The biological level of the third user may be compared to a predetermined threshold, to determine or detect lung-related or breathing-related conditions of the user. When no lung-related or breathing-related condition is determined or detected, the aerosol-generating device may continue to grant usage access to the third user.

Based on the estimated physical parameter and the biological state of the third user, the aerosol-generating device may also determine information associated with prescribed dosing of one or more substances, such as a nicotine content, a flavor content or even other medicament content, for the third user. The information associated with the prescribed dosing may be integrally stored in the aerosol-generating device, or remotely retrieved from a server, via a suitable communication network. Based on the information associated with the prescribed dosing, the aerosol-generating device may control the dosing of one or more substances for the third user. In an example, the prescribed dosing may relate to an increased substance content compared to a different substance content.

Therefore, it is advantageous that the aerosol-generating system is configured to selectively control a dosing of at least one substance content, such as nicotine content or flavor content of the consumable disposed in the aerosol-generating device, based on the state of the authorized user, such as a facial state and a biological state. It is further advantageous that the aerosol-generating system is configured to prevent over-consumption of certain products, via such features as the control of the consumption amount of the puffs, and/or the consumption duration of the aerosol-generating device, based on a biological level, such as the level of a lung condition, of the user.

The aerosol-generating system may also include other sensors, such as a temperature sensor and an inertial motion unit (IMU) sensor, to determine a usage behavior of the user. The temperature sensor may be configured to measure a temperature of the user. The IMU sensor may include an accelerometer and a gyroscope, which may be configured to measure an angular rate, a force and positional orientations of the user during usage of the aerosol-generating device. The usage behavior may correspond to at least one of a pattern of usage, such as a time, a duration of usage of the aerosol-generating device or a number of puffs of the aerosol-generating device. Based on the usage behavior, the estimated physical parameter and the biological state of the third user, the aerosol-generating system may improve the control of the prescribed dosing of one or more substances, such as a nicotine content, a flavor content or even other medicament content, for the third user.

FIG. 2 is a flow diagram showing a method 200 of controlling an aerosol-generating device. At least one step of method 200 is performed by a processor or a computing device. For example, the method 200 may be performed by an electronic device or an electronic system. The aerosol-generating system described with respect to FIG. 1 may perform the method 200. In an example, the method 200 is performed by a system, where any of the features for the aerosol-generating system described with respect to FIG. 1 can be part of the system on which the method 200 is performed.

The method 200 comprises, in step 210, capturing, by an image acquisition sensor, at least one image of a user. The user may try to use the aerosol-generating device. For example, the user may provide an input to the aerosol-generating device, which instructs the image acquisition sensor to capture the at least one image of a user. The image acquisition sensor may be part of the aerosol-generating device or remote from the aerosol-generating device.

In step 220, a sound generated by a respiration of the user is detected by means of an acoustic sensor. The acoustic sensor may be part of the aerosol-generating device. The steps 210 and 220 may be performed concurrently or subsequently. For example, step 220 may be performed after step 210. In an example, step 220 is performed after the at least one image of the user is processed by at least one processor. The at least one processor may determine whether a user is authorized to use the aerosol-generating device by performing a face-recognition method, and step 220 may only be performed when the user is authorized to use the aerosol-generating device.

In step 230, at least one of an access of the user to the aerosol-generating device and an amount of a substance and/or aerosol generate from an aerosol-forming substrate engaged with the aerosol-generating device is controlled based on at least one of the at least one image of the user and the detected sound. For example, the access of the user to the aerosol-generating device may be controlled by analyzing the at least one image of the user, the detected sound or both. Additionally or alternatively, the amount of the substance and/or aerosol generated from the aerosol-forming substrate engaged with the aerosol-generating device may be controlled by analyzing the at least one image of the user, the detected sound or both.

The analysis of the at least one image of the user may comprise determining, based on the at least one image of the user, at least one of an identity, a physical state and an emotional state of the user. The determined identity, the determined physical state or the determined emotional state may be used to control the access of the user to the aerosol-generating device or to control the amount of the substance and/or aerosol generated from the aerosol-forming substrate.

The analysis of the detected sound may comprise determining a biological status of the user based on the detected sound. The biological status may comprise at least one of a lung-related condition and a level of bronchitis of the user. The determined lung-related condition or the determined level of bronchitis of the user may be used to control the access of the user to the aerosol-generating device or to control the amount of the substance content of the aerosol-forming substrate. The biological status and the at least one of an identity, a physical state and an emotional state of the user may be determined by using a neural network.

In an example, the method 200 comprises determining an identity of the user based on the at least one captured image of the user, and verifying the identity of the user based on the detected sound, by determining that features derived from the detected sound correspond to features stored in a user profile. The features derived from the detected sound may be specific to the user.

For the purpose of the present description and of the appended claims, except where otherwise indicated, all numbers expressing amounts, quantities, percentages, and so forth are to be understood as being modified in all instances by the term “about”. Also, all ranges include the maximum and minimum points disclosed and include any intermediate ranges therein, which may or may not be specifically enumerated herein. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1.-15. (canceled)

16. An aerosol-generating system, comprising:
an aerosol-generating device configured to generate an aerosol from an aerosol-forming substrate;
an image acquisition sensor configured to capture at least one image of a user of the aerosol-generating device;
an acoustic sensor configured to detect a sound generated based on a respiration of the user; and
at least one processor configured to control, based on the at least one image of the user and the detected sound, at least one of:
an access of the user to the aerosol-generating device,
an amount of a substance generated from the aerosol-forming substrate, and
an amount aerosol generated from the aerosol-forming substrate.

17. The aerosol-generating system according to claim 16, wherein the at least one processor is further configured to determine, based on the detected sound, a biological status of the user.

18. The aerosol-generating system according to claim 17, wherein the biological status comprises at least one of a lung-related condition and a level of bronchitis of the user.

19. The aerosol-generating system according to claim 17, wherein the at least one processor is further configured to determine, based on the at least one image of the user, at least one of an identity, a physical state, and an emotional state of the user.

20. The aerosol-generating system according to claim 19, wherein the at least one of the access of the user to the aerosol-generating device and the amount of the substance or aerosol generated from the aerosol-forming substrate is controlled based on the biological status and the at least one of the identity, the physical state, and the emotional state of the user.

21. The aerosol-generating system according to claim 19,
wherein the at least one processor is further configured to determine a user profile based on at least one of the biological status and the at least one of the identity, the physical state, and the emotional state of the user, and
wherein the amount of the substance or aerosol generated from the aerosol-forming substrate is controlled in accordance with the user profile.

22. The aerosol-generating system according to claim 19, wherein the at least one processor is further configured to verify an identity of the user based on the biological status of the user.

23. The aerosol-generating system according to claim 19, wherein at least one of the biological status and the at least one of an identity, a physical state, and an emotional state of the user is determined by using a neural network.

24. The aerosol-generating system according to claim 16,
wherein the at least one processor is further configured to control the acoustic sensor to detect the sound based on an analysis of the at least one image of the user, and
wherein the analysis of the at least one image of the user comprises a determination whether the user is authorized.

25. The aerosol-generating system according to claim 16, wherein the at least one processor is further configured to control the access of the user to the aerosol-generating device by limiting usage of the aerosol-generating device to a predetermined number of puffs or a predetermined usage duration.

26. The aerosol-generating system according to claim 16, wherein the controlling the amount of a substance generated from the aerosol-forming substrate comprises increasing the amount of one substance generated from the aerosol-forming substrate with respect to another substance generated from the aerosol-generating substrate.

27. The aerosol-generating system according to claim 16,
further comprising at least one of a temperature sensor configured to measure a temperature of the user and an inertial motion unit (IMU) sensor,
wherein data of the IMU sensor is used to determine a usage behavior of the user, and
wherein the at least one of the access of the user to the aerosol-generating device and the amount of the substance and/or aerosol generated from the aerosol-forming substrate is controlled based on the at least one of the at least one image of the user and the detected sound, and at least one of the usage behavior of the user and the temperature.

28. The aerosol-generating system according to claim 16, wherein the at least one processor is further configured to:
determine whether the user is authorized to use the aerosol-generating device based on the at least one image of the user, and
when the user is unauthorized, at least one of:
lock the aerosol-generating device to prevent usage of the aerosol-generating device from the unauthorized user, and
provide an alert to an authorized user of the aerosol-generating device.

29. The aerosol-generating system according to claim 28,
wherein the alert comprises information associated with the unauthorized user attempt on the aerosol-generating device, and
wherein the alert comprises at least one of: an audible alert, a visual alert, an audio-visual alert, or a vibratory alert.

30. A method, comprising:
capturing, by an image acquisition sensor, at least one image of a user;
detecting, by an acoustic sensor, a sound generated based on a respiration of the user; and
controlling, based on the at least one image of the user and the detected sound, at least one of:
an access of the user to an aerosol-generating device,
an amount of a substance generated from an aerosol-forming substrate, and
an amount of aerosol generated from an aerosol-forming substrate.
